(19) United States
(12) Patent Application Publication
(10) Pub. No.: US 2015/0272741 A1
(43) Pub. Date: Oct. 1, 2015

(54) PRESS-FIT GLENOID WITH PERIPHERAL COMPRESSION PEGS

(71) Applicant: Biomet Manufacturing, LLC, Warsaw, IN (US)

(72) Inventors: Robert TAYLOR, Granger, IN (US); Thomas M. VANASSE, South Bend, IN (US)

(73) Assignee: Biomet Manufacturing, LLC, Warsaw, IN (US)

(21) Appl. No.: 14/226,051

(22) Filed: Mar. 26, 2014

(51) Int. Cl. A61F 2/40

(52) U.S. Cl. CPC A61F 2/4081

(57) ABSTRACT

A glenoid implant is provided and may include a body and a fixation member. The body may include an articular surface and a scapula-engaging surface opposite from the articular surface. The fixation member may extend from the scapula-engaging surface for fixing the glenoid implant to a scapula. The fixation member may include an annular fin having a peripheral surface that defines at least one dimple.

### FIELD

The present disclosure relates to an implant, and more particularly to a device and method for securing a glenoid implant to a glenoid through a press-fit configuration, including a press-fit configuration that further utilizes bone cement.

### BACKGROUND

This section provides background information related to the present disclosure which is not necessarily prior art.

Surgical procedures for repairing or reconstructing a joint may require securely fastening a surgical implant to a bone. For example, shoulder joint reconstruction may require fixing a glenoid implant to a scapula to reproduce or replicate a glenoid cavity on the scapula. The surgical implant may be securely fastened to the bone in a variety of ways, including mechanical fasteners, adhesive, and press-fit or frictionally-engaged pegs. When fastening the surgical implant to the bone with a press-fit or frictionally-engaged peg, it may be desirable to ensure a tight or otherwise secure interface between the peg and a bore or other aperture formed in the bone.

While known surgical implants have proven to be acceptable for their intended purposes, a continuous need for improvement in the relevant arts remains.

### SUMMARY

This section provides a general summary of the disclosure, and is not a comprehensive disclosure of its full scope or all of its features.

According to one particular aspect, the present disclosure provides a glenoid implant. The glenoid implant may include a body and a fixation member. The body may include an articular surface and a scapula-engaging surface opposite from the articular surface. The fixation member may extend from the scapula-engaging surface for fixing the glenoid implant to a scapula. The fixation member may include an annular fin having a peripheral surface that defines at least one dimple.

According to another particular aspect, the present disclosure provides a glenoid implant. The glenoid implant may include a body, a plurality of fixation members, and a central boss. The body may include an articular surface and a scapula-engaging surface opposite the articular surface. The plurality of fixation members may fix the glenoid implant to a scapula. Each of the plurality of fixation members may extend from a proximal end to a distal end and may include at least one annular fin defining a plurality of dimples in a peripheral surface of the annular fin. The distal end of each of the plurality of fixation members may include an annular flange. The central boss may extend from the scapula-engaging surface and may be disposed between the plurality of fixation members. The central boss may include a cylindrical outer surface. At least one of the dimples may be concentric with the cylindrical outer surface.

According to yet another particular aspect, the present disclosure provides an implant. The implant may include a body and a plurality of substantially cylindrical fixation members. The body may include a bone-engaging side. The plurality of fixation members may extend from the bone-engaging side for fixing the implant to a bone. Each of the plurality of fixation members may have a first diameter and may further include a first annular fin, a second annular fin, and a first annular groove. The first annular fin may include a first radially outward surface. The first radially outward surface may include at least one first concave portion and at least one first convex portion. The second annular fin may include a second radially outward surface. The second radially outward surface may include at least one second concave portion and at least one second convex portion. The first annular groove may be disposed between the first annular fin and the second annular fin. The first annular groove may define a second diameter of the fixation member that is smaller than the first diameter.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a first perspective view of an implant including an annular fin, in accordance with the principles of the present disclosure.

FIG. 2 is a second perspective view of the implant of FIG. 1.

FIG. 3A is a top view of the implant of FIG. 1.

FIG. 3B is a top view of another configuration of an annular fin, in accordance with the principles of the present disclosure.

FIG. 3C is a top view of another configuration of an annular fin, in accordance with the principles of the present disclosure.

FIG. 3D is a top view of yet another configuration of an annular fin, in accordance with the principles of the present disclosure.

FIG. 4 is a side view of the implant of FIG. 1.

FIG. 5 is an end view of the implant of FIG. 1, with a peripheral peg removed for clarity.

Corresponding reference numerals indicate corresponding parts throughout the several views of the drawings.

### DETAILED DESCRIPTION

Example embodiments will now be described more fully with reference to the accompanying drawings.

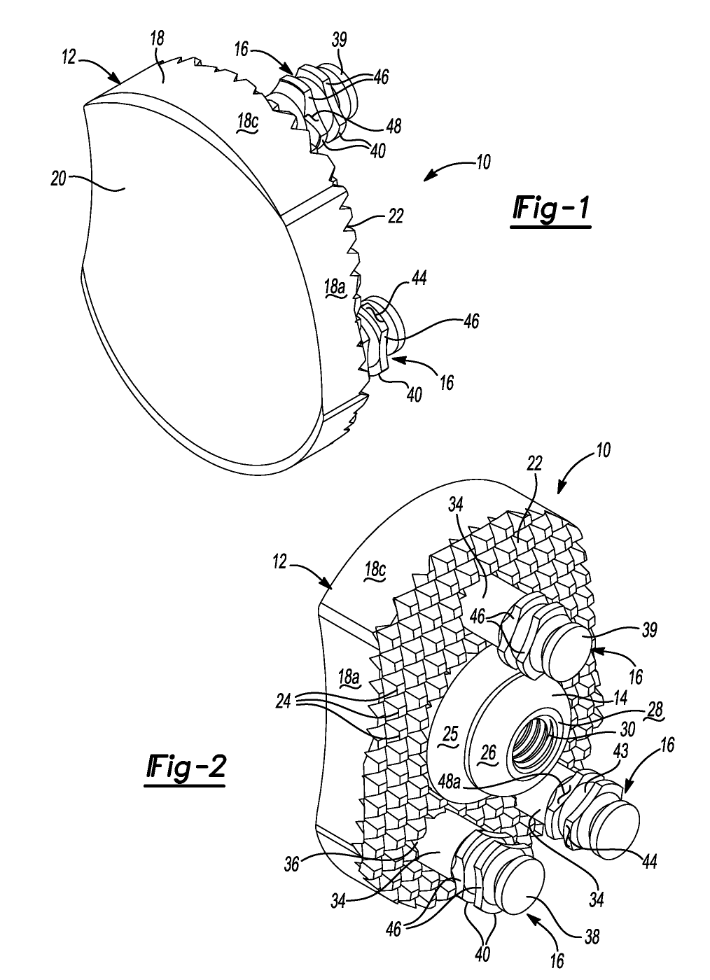

With reference to FIGS. 1-5, an implant constructed in accordance with the principles of the present disclosure is illustrated and identified at reference character 10. According to one exemplary use, the implant 10 may be a glenoid implant for use in shoulder joint replacement. In such case, the glenoid implant can replace or replicate an entire glenoid cavity or a portion thereof for anatomic shoulder joint replacements. The glenoid implant can also fill a defect in the glenoid cavity such as a void due to severe wear. It will also be appreciated, however, that the present teachings may be adapted to fix various implants to various bones.

The implant 10 may include a generally rectangular body 12 having a pear-shaped outline (by way of non-limiting example), a central fixation member or boss 14, and peripheral fixation members or pegs 16. The implant 10 can be formed from any biocompatible material, including, polymer, ceramic, metal or combinations thereof. The implant 10 can be formed using any suitable manufacturing technique, including machining, direct compression molding and/or additive manufacturing which enables forming multiple implants in a single build and decreases manufacturing time. Once formed, the implant 10 can be further processed (e.g., polished, blasted, machining) as desired. For example, the implant 10 can be polished for articulation with a humeral head made from polyethylene or another suitable material. Alternatively, polyethylene can be molded over or pressed onto the body 12 for articulation with a metal humeral head.

The implant 10 may be configured to be fixed to a bone without using fixation hardware such as bone screws. As will be described in more detail below, the central boss 14 and/or the peripheral pegs 16 can be press fit into holes formed in the bone to fix the implant 10 to the bone. In addition, the implant 10 may be configured to receive bone cement to fix the peripheral pegs 16 within corresponding bores or holes in the bone.

The body 12 may include a peripheral surface 18, an articular side 20, and a bone-engaging side 22 opposite from the articular side 20. The peripheral surface 18 may include superior and inferior portions 18a, 18b that are flat or slightly rounded (e.g., concave), and anterior and posterior portions 18c, 18d that are rounded (e.g., convex). The peripheral surface 18 can be patient-specific and can match or replicate a peripheral surface of a glenoid cavity of a specific patient. The central boss 14 and the peripheral pegs 16 may extend from the bone-engaging side 22 of the body 12. As illustrated, the central boss 14 may be located central to, or otherwise between, the peripheral pegs 16, and may define a longitudinal axis 23. Although the implant 10 is shown with three peripheral pegs 16 surrounding the central boss 14, the implant 10 can include more or less than three peripheral pegs within the scope of the present disclosure.

The articular side 20 of the implant 10 may be configured to partially receive and nestingly engage or articulate with the humeral head. For example, the articular side 20 can include a concave hemispherically shaped surface that closely conforms as a mirror-image of a complementary surface of the humeral head. The humeral head can be part of a natural humerus of a specific patient, or the humeral head can be part of a humeral implant.

The bone-engaging side 22 may include a plurality of protrusions or teeth 24 extending therefrom. The teeth 24 may be shaped as pyramidal frustoms. In one configuration, the teeth 24 may be shaped as truncated square pyramids. The teeth 24 may be arranged in a grid or array of orthogonally disposed rows, helping bone cement to flow or otherwise disburse between the bone and the bone-engaging side.

The central boss 14 and/or the peripheral pegs 16 can be formed monolithically with the body 12 or separate from the body 12. In regard to the latter, at least one of the central boss 14 and/or the peripheral pegs 16 can be formed as separate components and mechanically joined, press fit, or threaded into an aperture formed in the bone-engaging side 22 of the body 12. The central boss 14 may be substantially cylindrical, including a tapered or concavely radiused surface 25 adjacent the bone-engaging side 22 and a convexly radiused surface 26 adjacent a substantially planar end surface 28 of the central boss 14. The end surface 28 may include a threaded bore or aperture 30 for receiving an insert or removable central peg (not shown). The central peg may be coated with a porous material for improving the in-growth of bone into the aperture 30, and thus improve the stability and fixation of the implant 10 relative to the bone by ensuring a secure connection between the central boss 14 and a corresponding hole in the bone.

The peripheral pegs 16 may include a substantially cylindrical body 34 extending between a proximal end 36 and a distal end 38 and having a diameter D1. The proximal end 36 may be adjacent to the bone-engaging side 22 of the body. The peripheral pegs 16 may also include an annular flange 39 and at least one annular fin 40 extending from a peripheral surface of the body 34. In one configuration, each peripheral peg 16 includes two annular fins 40. It will be appreciated, however, that each peg 16 may include more or less than two annular fins 40 within the scope of the present disclosure.

The annular flange 39 may be formed adjacent to the distal end 38 of the peg 16 and may be generally circular, including a diameter D2. The diameter D2 may be substantially equal to the diameter D1 of the body 34. In this regard, it will be appreciated that the annular flange 39 may be defined in part by a first annular groove 41 formed in the body 34 of the peg 16. The first annular groove 41 may have a diameter D3 that is less than the diameters D1 and D2 of the body 34 and the annular flange 39, respectively. As will be described in more detail below, the annular flange 39 and the first annular groove 41, including the diameters D2 and D3, respectively, can improve the alignment of the peg 16 within the bore formed in the bone and can improve the retention of bone cement between the peg 16 and the bone.

The annular fins 40 may be formed between the proximal and distal ends 36, 38 of the peg 16. As illustrated in FIG. 5, the annular fins 40 may be located at distances X1 and X2 from the distal end 38 of the peg 16. The annular fin 40 may have an axially extending thickness T1 and a radially extending length T2 as measured from an outer peripheral surface of the cylindrical body 34. In one configuration, the radially extending length T2 may be approximately ten percent greater than the axially extending depth T1. The ratio of the radially extending length T2 to the axially extending thickness T1 can help to improve the flexural characteristics of the annular fin 40.

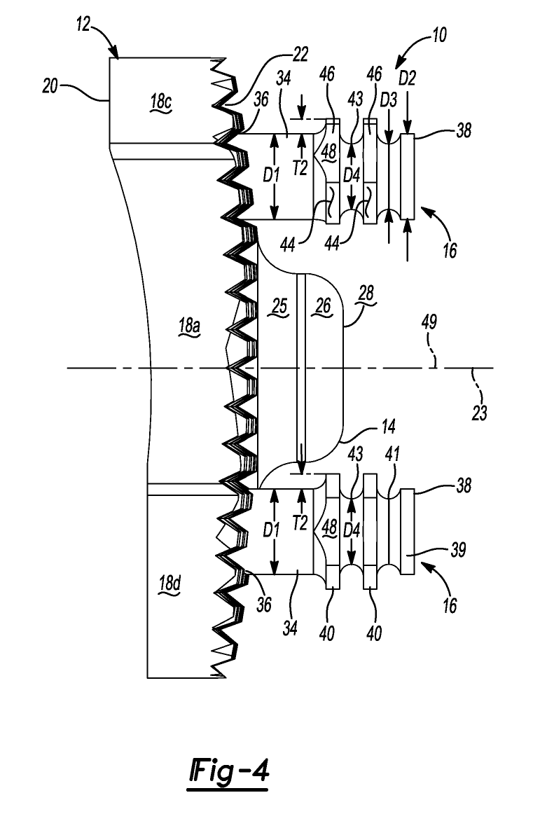

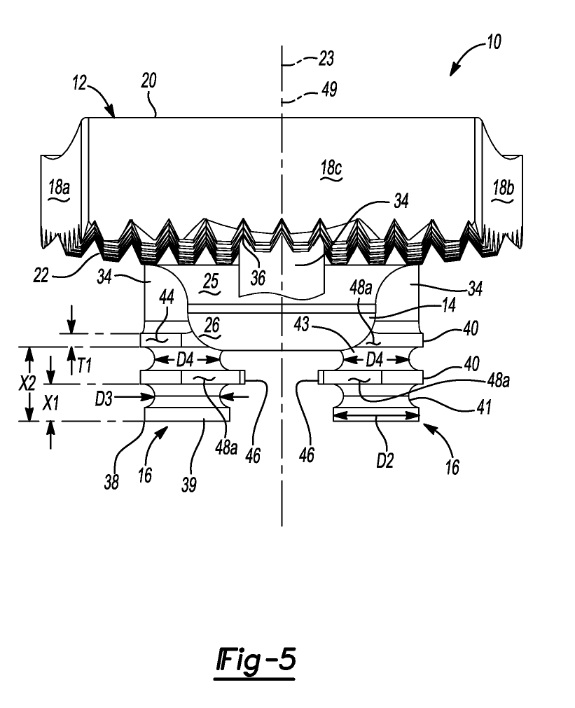

As illustrated in FIGS. 4 and 5, a second annular groove 43, having a fourth diameter D4, may be formed between consecutive annular fins 40. The fourth diameter D4 may be less than the first diameter D1 of the body 34 and substantially equal to the third diameter D3 of the first annular groove 41. In one configuration, the diameter D1 plus two times the length T2 may be approximately eighty percent greater than the diameter D4. In this way, the second annular groove 43 can improve the retention of bone cement between the peg 16 and the bone. As will be explained in more detail below, the second annular groove 43 can also reduce the stiffness of the annular fins 40, thus allowing the fins 40 to flex when the implant 10 is assembled into the bone.

The annular fin 40 may include a substantially convex peripheral surface 44 defining a plurality of cutout and/or shaped portions generally referred to herein as scallops or dimples 46. The dimples 46 may include a substantially concave surface 48. In one configuration, the peripheral surface 44 includes three equally spaced-apart dimples 46, such that the peripheral surface 44 defines a substantially scalloped profile or trilobular shape, including alternating convex portions formed by the peripheral surface 44 and by the concave surfaces 48 of the dimples 46. At least one concave surface 48a may define an arc having a central axis 49. As illustrated in FIGS. 4 and 5, the axis 49 may be substantially aligned with the longitudinal axis 23 of the central boss 14, such that the concave surface 48a is substantially concentric with the central boss 14. The concentricity of the concave surface 48a and the boss 14 can improve the manufacturability of the implant 10 by ensuring a smaller number of machining operations or other similar manufacturing processes.

While the peripheral surface 44 is shown with equally sized and space dimples 46, it will also be appreciated that the peripheral surface 44 may include different sized and spaced dimples 46 within the scope of the present disclosure. In this regard, a first of the annular fins 40 may include a first plurality of dimples 46 having a first size and shape, and another of the annular fins 40 may include a second plurality of dimples 46 having a second size and shape different than the first size and shape.

The scalloped profile formed by the dimples 46 may decrease the stiffness of the annular fin 40, and thus allow the annular fin 40 to flex in response to an axial load or force. As will be explained in more detail below, the dimples 46 may also allow for the flow of bone cement between the bone and the annular fin 40 when the implant 10 is in an assembled configuration. In this regard, it will be appreciated that the size, shape and location of the dimples 46 can be varied to adjust the flow characteristics of the bone cement and/or to adjust the flexural characteristics of the annular fin 40.

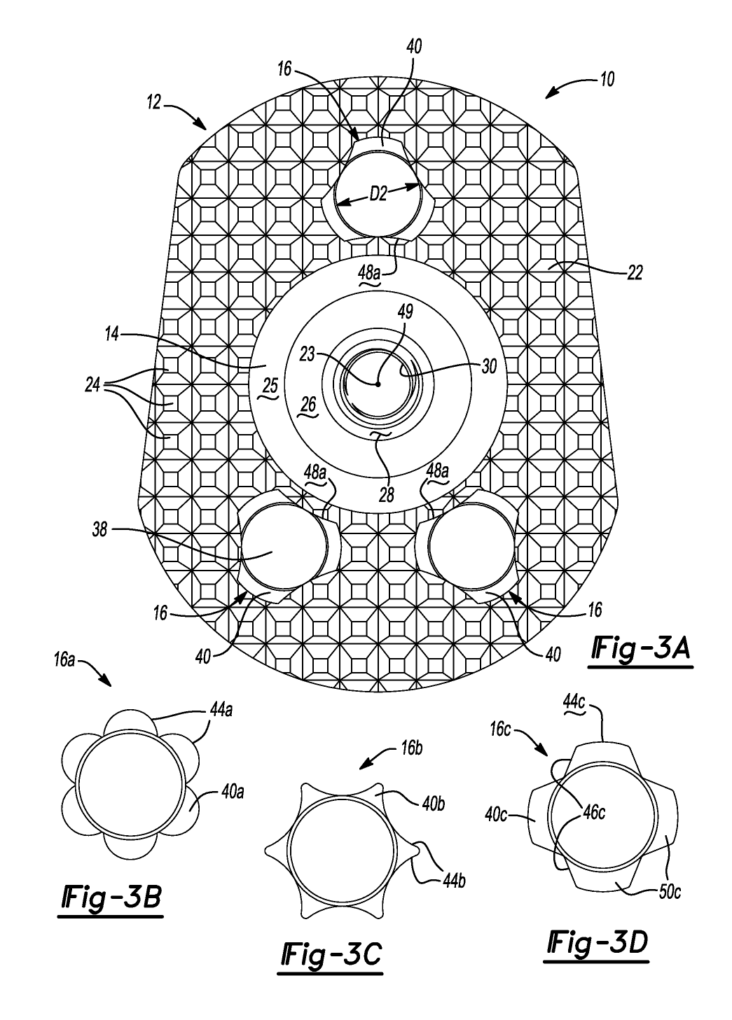

With reference to FIGS. 3B, 3C and 3D, other configurations of a peg 16 and an annular fin 40 are shown. The pegs 16a, 16b, 16c and annular fins 40a, 40b, 40c shown in FIGS. 3B, 3C and 3D, respectively, may be substantially similar to the peg 16 and annular fin 40 shown in FIG. 3A, except as otherwise provided herein. With reference to the peg 16a illustrated in FIG. 3B, the annular fin 40a may include a series of consecutive lobes defined by a convex peripheral edge or surface 44a. With reference to the peg 16b illustrated in FIG. 3C, the annular fin 40b may include a series of consecutive lobes defined by a concave peripheral edge or surface 44b. Finally with reference to the peg 16c illustrated in FIG. 3D, the annular fin 40c may include at least one V-shaped cutout or recess 46c, such that a peripheral surface 44c defines an alternating pattern of recesses 46c and lobes 50c.

An example method of repairing a bone, such as a glenoid cavity of a scapula (not shown), will now be described. First, a surgeon may drill or otherwise form holes in the scapula. A first hole can be sized for receiving the central boss 14, which may also include the central peg (not shown). A plurality of second holes can be sized for receiving the peripheral pegs 16. In this regard, the second holes may have a fifth diameter D5. The fifth diameter D5 may be substantially equal to or slightly greater than the first diameter D1 of the body 34 and equal to or slightly greater than the second diameter D2 of the annular flange 39.

The implant 10 may be placed within the glenoid cavity such that the peripheral pegs 16 are aligned with the second holes and the central boss 14 is aligned with the first hole. A force may be applied to the implant 10 such that the annular fins 40 engage the second holes of the scapula in a press-fit configuration. In this regard, it will be appreciated that the annular fin 40 may deform or otherwise flex between consecutive dimples 46, to ensure initial stability and a secure fit between the implant 10 and the scapula. Bone cement may be inserted into the prepared peripheral holes in the scapula. The bone cement may flow between the dimples 46 and the scapula, as well as between the body 34 and the scapula, to ensure that the implant 10 is adequately secured to the scapula.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A glenoid implant, comprising:
a body having an articular surface and a scapula-engaging surface opposite from the articular surface; and
a fixation member extending from the scapula-engaging surface for fixing the glenoid implant to a scapula, the fixation member including an annular fin having a peripheral surface, the peripheral surface defining at least one dimple.

2. The glenoid implant of claim 1, further comprising a plurality of fixation members and central boss extending from the scapula-engaging surface, the central boss disposed between the plurality of fixation members.

3. The glenoid implant of claim 2, wherein the central boss includes a cylindrical outer surface, and wherein the at least one dimple is concentric with the cylindrical outer surface.

4. The glenoid implant of claim 3, wherein the implant includes three fixation members, each fixation member defining a dimple concentric with the cylindrical outer surface of the central boss.

5. The glenoid implant of claim 2, wherein each of the plurality of fixation members includes a first annular fin defining a first dimple concentric with the central boss and a second annular fin defining a second dimple concentric with the central boss.

6. The glenoid implant of claim 1, wherein the fixation member includes a first annular fin and a second annular fin, and wherein the fixation member defines an annular groove disposed between the first annular fin and the second annular fin.

7. The glenoid implant of claim 1, wherein the fixation member extends from a proximal end to a distal end, and wherein the distal end includes an annular flange.

8. The glenoid implant of claim 7, wherein the fixation member includes a substantially cylindrical body having a first diameter D1, and wherein the annular flange defines a substantially circular perimeter having a second diameter D2.

9. The glenoid implant of claim 8, wherein the first diameter D1 is substantially equal to the second diameter D2.

10. The glenoid implant of claim 7, wherein the fixation member defines an annular groove disposed between the annular flange and the annular fin.

11. A glenoid implant, comprising:
a body having an articular surface and a scapula-engaging surface opposite from the articular surface;
a plurality of fixation members for fixing the glenoid implant to a scapula, each of the plurality of fixation members extending from a proximal end to a distal end and including at least one annular fin defining a plurality of dimples in a peripheral surface thereof, the distal end including an annular flange; and
a central boss extending from the scapula-engaging surface, the central boss disposed between the plurality of fixation members and including a cylindrical outer surface,
wherein at least one of the dimples is concentric with the cylindrical outer surface.

12. The glenoid implant of claim 11, wherein each of the plurality of fixation members includes a substantially cylindrical body having a first diameter D1, and wherein the annular flange defines a substantially circular perimeter having a second diameter D2.

13. The glenoid implant of claim 12, wherein the first diameter D1 is substantially equal to the second diameter D2.

14. The glenoid implant of claim 11, wherein each of the plurality of fixation members defines an annular groove disposed between the annular flange and the annular fin.

15. An implant comprising:
a body having a bone-engaging side; and
a plurality of substantially cylindrical fixation members extending from the bone-engaging side for fixing the implant to a bone, each of the plurality of fixation members having a first diameter and further including:
a first annular fin having a first radially outward surface, the first radially outward surface including at least one first concave portion and at least one first convex portion,
a second annular fin having a second radially outward surface, the second radially outward surface including at least one second concave portion and at least one second convex portion, and
a first annular groove disposed between the first annular fin and the second annular fin, the first annular groove defining a second diameter of the fixation member, the second diameter smaller than the first diameter.

16. The implant of claim 15, wherein the first and second annular fins each include a first shape, the first shape defining a substantially trilobular peripheral surface.

17. The implant of claim 15, wherein a first of the at least one first concave portions is substantially concentric to a first of the at least one second concave portions.

18. The implant of claim 15, further comprising a central boss disposed between the fixation members, wherein the central boss includes a substantially cylindrical outer surface.

19. The implant of claim 18, wherein the central boss is substantially concentric with a first of the at least one first concave portions and with a first of the at least one second concave portions.

20. The implant of claim 15, wherein at least one of the fixation members further comprises a second annular groove disposed between the first annular fin and a distal end of the fixation member, the second annular groove defining a third diameter of the fixation member, the third diameter smaller than the first diameter.
